(19) United States
(12) Patent Application Publication
(10) Pub. No.: US 2025/0090668 A1
(43) Pub. Date: Mar. 20, 2025

(54) GLYCOL COMPOSITIONS INCLUDING TOCOPHEROLS

(71) Applicant: Dow Global Technologies LLC, Midland, MI (US)

(72) Inventor: Caroline Woelfle-Gupta, Midland, MI (US)

(21) Appl. No.: 18/727,727

(22) PCT Filed: Jan. 13, 2023
(86) PCT No.: PCT/US2023/010727

§ 371 Date: Jul. 10, 2024

Related U.S. Application Data
(60) Provisional application No. 63/299,610, filed on Jan. 14, 2022.

(51) Int. Cl. A61K 47/22; A61K 8/67; A61K 8/86; A61K 47/10

(52) U.S. Cl. CPC A61K 47/22; A61K 8/678; A61K 8/86; A61K 47/10

(57) ABSTRACT

A glycol composition comprises a glycol and a tocopherol blend comprising D-alpha tocopherol and one or more of D-beta tocopherol, D-gamma tocopherol and D-delta tocopherol, wherein the D-alpha tocopherol is less than 50 wt % of the combined D-alpha tocopherol, D-beta tocopherol, D-gamma tocopherol and D-delta tocopherol.

### BACKGROUND

### Field of the Disclosure

The present disclosure is directed to glycol compositions and more specifically to glycol compositions comprising tocopherols.

### INTRODUCTION

Glycols based on alkyl ethers come in a variety of forms. For example, glycols may be a low molecular weight solvent or may include polymerized moieties of the alkyl ethers (e.g., polyethylene glycol or propylene glycol). During storage, glycols may oxidize and/or undergo one or more reactions that produce impurities such as formaldehyde. For certain glycol applications, the United States Food and Drug Administration has established a maximum threshold of formaldehyde formation during the glycol storage life. As such, minimizing the formaldehyde formation during the storage is important. One or more antioxidants may be included in the glycol to prevent degradation and formaldehyde formation during storage.

Tocopherols exhibit an antioxidative activity via hydrogen donation to a free radical and subsequent formation of a complex between the radical and the tocopherol radical. The use of tocopherols in glycol compositions as an antioxidant is known. For example, U.S. Pat. No. 5,695,689A (“the '689 patent”) discloses the use of D-alpha tocopherol and/or D-beta tocopherol with a polyether polyol having a molecular weight of from about 300 to about 10,000 to stabilize the polyether polyol. The '689 patent is silent as to which tocopherol disclosed is the most effective or the relative amounts. Other instances of tocopherols being used as antioxidants exist. For example, U.S. Pat. No. 5,218,008 (“the '008 patent”) discloses the use of tocopherol mixtures as an antioxidant for polyether compounds. The '008 patent explains that in terms of antioxidant effectiveness the “[m]ost preferred is a α-tocopherol or a mixture of tocopherols of which α-tocopherol constituents at least 50%, more preferably at least 80% by weight.”

In view of the foregoing, it would be surprising to discover a useful glycol composition utilizing a tocopherol blend in which D-alpha tocopherol is less than 50 wt % of the tocopherols in the tocopherol blend.

### SUMMARY OF THE DISCLOSURE

The present disclosure provides a useful glycol composition utilizing a tocopherol blend in which D-alpha tocopherol is less than 50 wt % of the tocopherols in the tocopherol blend.

The inventor of the present disclosure has surprisingly discovered that glycol compositions including a tocopherol blend exhibit a reduction in the total tocopherol content when exposed to 316 stainless steel under decreasing temperature conditions. Such conditions will exist during transport of the glycol composition via rail or truck. The reduced total tocopherol content experience by the glycol composition will tend to increase formaldehyde levels overtime (i.e., because there is less antioxidant present) and may lead to the glycol composition failing one or more certifications due to formaldehyde levels. As such, the reduction of tocopherol content should be avoided.

The inventor of the present disclosure has also discovered that glycol composition including a tocopherol blend in which D-alpha tocopherol is less than 50 with of the tocopherols (i.e., the combined D-alpha tocopherol, D-beta tocopherol, D-gamma tocopherol and D-delta tocopherol) in the tocopherol blend exhibits reduced antioxidant fade (i.e., total tocopherol level reduction) compared tocopherols blends where 50% or greater of the tocopherols are D-alpha tocopherol.

The present disclosure is particularly advantageous in the formation of glycol compositions.

According to a first feature of the present disclosure, a glycol composition comprises a glycol and a tocopherol blend comprising D-alpha tocopherol and one or more of D-beta tocopherol, D-gamma tocopherol and D-delta tocopherol, wherein the D-alpha tocopherol is less than 50 wt % of the combined D-alpha tocopherol, D-beta tocopherol, D-gamma tocopherol and D-delta tocopherol.

According to a second feature of the present disclosure, the glycol is 80.000 wt % to 99.999 wt % of the total weight of the glycol composition.

According to a third feature of the present disclosure, the glycol is a polyalkylene oxide having a weight average molecular weight of 400 g/mol to 10,000,000 g/mol as measured according to gel permeation chromatography.

According to a fourth feature of the present disclosure, the glycol is a polyalkylene oxide having a weight average molecular weight of 2,000 g/mol to 5,000 g/mol as measured according to gel permeation chromatography.

According to a fifth feature of the present disclosure, the glycol is polyethylene glycol.

According to a sixth feature of the present disclosure, the glycol composition comprises 0.01 wt % or less of the tocopherol blend based on the total weight of the glycol composition.

According to a seventh feature of the present disclosure, the glycol composition comprises 0.001 wt % or greater of the tocopherol blend based on the total weight of the glycol composition.

According to an eighth feature of the present disclosure, the D-alpha tocopherol is 0 wt % to 40 wt % of the combined D-alpha tocopherol, D-beta tocopherol, D-gamma tocopherol and D-delta tocopherol.

According to a ninth feature of the present disclosure, the D-alpha tocopherol is 10 wt % to 30 wt % of the combined D-alpha tocopherol, D-beta tocopherol, D-gamma tocopherol and D-delta tocopherol.

According to a tenth feature of the present disclosure, a formulation, comprises 0.1 wt % to 99 wt % of the glycol composition based on the total weight of the formulation.

### DETAILED DESCRIPTION

As used herein, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

All ranges include endpoints unless otherwise stated.

As used herein, the term weight percent (“wt %”) designates the percentage by weight a component is of a total weight of the glycol composition unless otherwise specified.

As used herein, Chemical Abstract Services registration numbers (“CAS #”) refer to the unique numeric identifier as most recently assigned as of the priority date of this document to a chemical compound by the Chemical Abstracts Service.

### Glycol Compositions

The present disclosure is generally directed to glycol compositions. The glycol compositions comprise a glycol and a tocopherol blend. The glycol composition may comprise one or more antioxidants or other additives designed to alter a property or characteristic of the glycol composition.

### Glycol

The glycol composition comprises one or more glycols. As used herein, a glycol is defined as a chemical compound that comprises 2 or more hydroxyl groups. The glycol may be a low molecular weight compound selected from the group consisting of dipropylene glycol ethyl ether, tripropylene glycol ethyl ether, propylene glycol isopropyl ether, dipropylene glycol isopropyl ether, tripropylene glycol isopropyl ether, propylene glycol n-propyl ether, dipropylene glycol n-propyl ether, tripropylene glycol n-propyl ether, propylene glycol t-butyl ether, dipropylene glycol t-butyl ether, tripropylene glycol t-butyl ether, propylene glycol n-butyl ether, dipropylene glycol n-butyl ether, tripropylene glycol n-butyl ether, propylene glycol n-pentyl ether, propylene glycol n-hexyl ether, butylene glycol methyl ether, dibutylene glycol methyl ether, ethylene glycol n-butyl ether, ethylene glycol n-pentyl ether, ethylene glycol n-hexyl ether, ethylene glycol n-heptyl ether, ethylene glycol 2-ethylhexyl ether, diethylene glycol n-hexyl ether, propylene glycol methyl ether acetate, propylene glycol ethyl ether acetate, propylene glycol isopropyl ether acetate, propylene glycol n-propyl ether acetate, propylene glycol n-butyl ether acetate, dipropylene glycol methyl ether acetate, dipropylene glycol ethyl ether acetate, ethylene glycol n-butyl ether acetate, propylene glycol isobutyl ether, dipropylene glycol isobutyl ether, tripropylene glycol isobutyl ether, ethylene glycol t-butyl ether, ethylene glycol isobutyl ether, ethylene glycol ethyl ether acetate, ethylene glycol isobutyl ether acetate, diethylene glycol ethyl ether acetate, dipropylene glycol dimethyl ether, and diethylene glycol n-butyl ether acetate and combinations thereof.

Additionally or alternatively, the glycol may be a higher molecular weight compound that comprises a polymerized polyalkylene oxide. The polyalkyene oxide may be one or more of polyethylene oxide, polypropylene oxide, and polybutylene oxide. In such examples, the glycol may be a polyethylene glycol, polypropylene glycol, polybutylene oxide, and combinations thereof. The polyalkylene oxide glycol may have a weight average molecular weight of 400 grams per mole (“g/mol”) to 10,000,000 g/mol as measured according to gel permeation chromatography. For example, the polyalkylene oxide may have a weight average molecular weight of 400 g/mol or greater, or 500 g/mol or greater, or 1,000 g/mol or greater, or 1,500 g/mol or greater, or 2,000 g/mol or greater, or 2,500 g/mol or greater, or 3,000 g/mol or greater, or 3,500 g/mol or greater, or 4,000 g/mol or greater, or 4,500 g/mol or greater, or 5,000 g/mol or greater, or 5,500 g/mol or greater, or 6,000 g/mol or greater, or 6,500 g/mol or greater, or 7,000 g/mol or greater, or 7,500 g/mol or greater, or 8,000 g/mol or greater, or 8,500 g/mol or greater, or 9,000 g/mol or greater, or 9,500 g/mol or greater, or 10,000 g/mol or greater, or 10,500 g/mol or greater, or 11,000 g/mol or greater, or 11,500 g/mol or greater, or 12,000 g/mol or greater, or 12,500 g/mol or greater, or 13,000 g/mol or greater, or 13,500 g/mol or greater, or 14,000 g/mol or greater, or 14,500 g/mol or greater, or 15,000 g/mol or greater, or 20,000 g/mol or greater, or 40,000 g/mol or greater, or 60,000 g/mol or greater, or 80,000 g/mol or greater, or 100,000 g/mol or greater, or 500,000 g/mol or greater, or 1,000,000 g/mol or greater, or 5,000,000 g/mol or greater, while at the same time, 10,000,000 g/mol or less, or 5,000,000 g/mol or less, or 1,000,000 g/mol or less, or 500,000 g/mol or less, or 100,000 g/mol or less, or 50,000 g/mol or less, or 25,000 g/mol or less, or 20,000 g/mol or less, or 15,000 g/mol or less, or 14,500 g/mol or less, or 14,000 g/mol or less, or 13,500 g/mol or less, or 13,000 g/mol or less, or 12,500 g/mol or less, or 12,000 g/mol or less, or 11,500 g/mol or less, or 11,000 g/mol or less, or 10,500 g/mol or less, or 10,000 g/mol or less, or 9,500 g/mol or less or 9,000 g/mol or less, or 8,500 g/mol or less or 8,000 g/mol or less, or 7,500 g/mol or less or 7,000 g/mol or less, or 6,500 g/mol or less or 6,000 g/mol or less, or 5,500 g/mol or less or 5,000 g/mol or less, or 4,500 g/mol or less or 4,000 g/mol or less, or 3,500 g/mol or less or 3,000 g/mol or less, or 2,500 g/mol or less or 2,000 g/mol or less, or 1,500 g/mol or less or 1,000 g/mol or less, 500 g/mol or less as measured according to gel permeation chromatography.

The glycol composition comprises 80.000 wt % to 99.999 wt % of the glycol based on the total weight of the glycol composition. For example, the glycol composition may comprise 80.000 wt % or greater, or 85.000 wt % or greater, or 90.000 wt % or greater, or 95.000 wt % or greater, or 99.000 wt % or greater, or 99.070 wt % or greater, while at the same time, 99.999 wt % or less, or 95.000 wt % or less, or 90.000 wt % or less, or 85.000 wt % or less of the glycol based on the total weight of the glycol composition.

### Tocopherol Blend

The tocopherol blend comprises D-alpha tocopherol (CAS #59-02-9) and one or more of D-beta tocopherol (CAS #16698-35-4), D-gamma tocopherol (CAS #54-28-4) and D-delta tocopherol (CAS #119-13-1). In the tocopherol blend, the D-alpha tocopherol is less than 50 wt % of the combined D-alpha tocopherol, D-beta tocopherol, D-gamma tocopherol and D-delta tocopherol. For example, the D-alpha tocopherol may be 0.1 wt % or greater, or 1 wt % or greater, or 5 wt % or greater, or 10 wt % or greater, or 14 wt % or greater, or 15 wt % or greater, or 20 wt % or greater, or 25 wt % or greater, or 30 wt % or greater, or 35 wt % or greater, or 40 wt % or greater, or 45 wt % or greater, or 49.9 wt % or greater, while at the same time, less than 50 wt %, or 49.9 wt % or less, or 45 wt % or less, or 40 wt % or less, or 35 wt % or less, or 30 wt % or less, or 25 wt % or less, or 20 wt % or less, or 15 wt % or less, or 14 wt % or less, or 10 wt % or less, or 5 wt % or less, or 1 wt % or less based on the combined weight of the D-alpha tocopherol, D-beta tocopherol, D-gamma tocopherol and D-delta tocopherol.

The tocopherol composition may independently comprise each of the D-beta tocopherol, D-gamma tocopherol and D-delta tocopherol in an amount ranging from 1 wt % to 75 wt % based on the combined weight of the D-alpha tocopherol, D-beta tocopherol, D-gamma tocopherol and D-delta tocopherol. For example, each of the D-beta tocopherol, D-gamma tocopherol and D-delta tocopherol may independently be present in the tocopherol blend in an amount of 1 wt % or greater, or 2 wt % or greater, or 5 wt % or greater, or 10 wt % or greater, or 15 wt % or greater, or 20 wt % or greater, or 25 wt % or greater, or 30 wt % or greater, or 35 wt % or greater, or 40 wt % or greater, or 45 wt % or greater, or 50 wt % or greater, or 55 wt % or greater, or 60 wt % or greater, or 65 wt % or greater, or 70 wt % or greater, while at the same time, 75 wt % or less, or 70 wt % or less, or 65 wt % or less, or 60 wt % or less, or 55 wt % or less, or 50 wt % or less, or 45 wt % or less, or 40 wt % or less, or 35 wt % or less, or 30 wt % or less, or 25 wt % or less, or 20 wt % or less, or 15 wt % or less, or 10 wt % or less, or 05 wt % or less, or 2 wt % or less based on the combined weight of the D-alpha tocopherol, D-beta tocopherol, D-gamma tocopherol and D-delta tocopherol. One example of a commercially suitable tocopherol blend is COVI-OX™ T-90 from BASF, Ludwigshafen, Germany.

The glycol composition comprises from 0.001 wt % to 0.01 wt % of the tocopherol composition based on the total weight of the glycol composition. For example, the glycol composition may comprise 0.001 wt % or greater, or 0.002 wt % or greater, or 0.003 wt % or greater, or 0.004 wt % or greater, or 0.005 wt % or greater, or 0.006 wt % or greater, or 0.007 wt % or greater, or 0.008 wt % or greater, or 0.009 wt % or greater, while at the same time, 0.01 wt % or less, or 0.009 wt % or less, or 0.008 wt % or less, or 0.007 wt % or less, or 0.006 wt % or less, or 0.005 wt % or less, or 0.004 wt % or less, or 0.003 wt % or less, or 0.002 wt % or less of the tocopherol blend based on the total weight of the glycol composition.

### Formulation

The glycol composition may be used in one or more formulations. The formulation may be a cleaning composition, a cosmetics composition, industrial composition, a pharmaceutical, a personal care material, a food additive or other material comprising the glycol composition. The formulation comprises 0.1 wt % to 99 wt % of the glycol composition based on the total weight of the formulation. For example, the formulation may comprise 0.1 wt % or greater, or 1 wt % or greater, or 5 wt % or greater, or 10 wt % or greater, or 20 wt % or greater, or 30 wt % or greater, or 40 wt % or greater, or 50 wt % or greater, or 60 wt % or greater, or 70 wt % or greater, or 80 wt % or greater, or 90 wt % or greater, while at the same time, 99 wt % or less, or 90 wt % or less, or 80 wt % or less, or 70 wt % or less, or 60 wt % or less, or 50 wt % or less, or 40 wt % or less, or 30 wt % or less, or 20 wt % or less, or 10 wt % or less, or 1 wt % or less of the glycol composition based on the total weight of the formulation.

### Examples

### Materials

The following materials were used in the examples.

PEG is a polyethylene oxide glycol having a weight average molecular weight of 3,350 g/mol as measured according to gel permeation chromatography. The polyethylene glycol is commercially available from The Dow Chemical Company, Midland, Michigan.

AO1 is a tocopherol blend having greater than 90 wt % tocopherols based on the total weight of AO1 and having a composition of 14 wt % D-alpha tocopherol, 2 wt % D-beta tocopherol, 60 wt % D-gamma tocopherol and 24 wt % D-delta tocopherol, where the weight percentages for each individual tocopherol is based on the total weight of the combined D-alpha tocopherol, D-beta tocopherol, D-gamma tocopherol and D-delta tocopherol. AO1 is commercially available as COVI-OX™ T-90 from Kensing LLC, Kankakee, Illinois.

AO2 is a tocopherol blend having 537 mg/g of D-alpha tocopherol and 190 mg/g of non D-alpha tocopherols (D-beta tocopherol, D-gamma tocopherol, D-delta tocopherol). AO2 is commercially available as COVITOL™ F-80/20M from Kensing LLC, Kankakee, Illinois.

AO3 is a tocopherol blend having-960 mg/g of D-alpha tocopherol and 0 mg/g of non D-alpha tocopherols (D-beta tocopherol, D-gamma tocopherol, D-delta tocopherol). AO3 is commercially available as COVITOL™ F-1490 from Kensing LLC, Kankakee, Illinois.

### Sample Preparation

The examples were prepared by first making a masterbatch of PEG containing 0.1 wt % of the chosen antioxidant based on the total weight of the masterbatch. The different PEG and antioxidant masterbatches were made by weighing out 0.1 gram (“g”) (+/−0.008 g) of the chosen antioxidant in a 100-milliliter vial, followed by 99.9 g of the PEG. Once both materials were weighed, the vials were placed in an oven at 70° C. until fully melted. Once melted, the formulations were mixed for twenty minutes on an overhead mixer maintained at 65° C. and at a speed of 330 revolutions per minute (“RPM”). To make the formulations containing 0.002 wt % of antioxidant, 2 g of the melted masterbatch was added to 98 g of melted PEG and mixed at 65° C. for 40 minutes on an overhead stirrer using a speed of 330 rpm. Similarly, to make the formulations containing 0.005 wt % of antioxidant, 5 g of the melted masterbatch was added to 95 g of melted PEG and mixed at 65° C. for 40 minutes on an overhead mixer at a speed of 330 RPM speed. Two replicates per concentration and per antioxidant were made. The compositions of the inventive examples (“IE”) and the comparative examples (“CE”) are provided in Table 1.

TABLE 1

| | | AO1 | | |
|---|---|---|---|---|
| | PEG | (wt | AO2 | AO3 |
| | (wt %) | %) | (wt %) | (wt %) |
| IE1 | 99.998 | 0.002 | 0 | 0 |
| IE2 | 99.995 | 0.005 | 0 | 0 |
| CE1 | 99.998 | 0 | 0.002 | 0 |
| CE2 | 99.995 | 0 | 0.005 | 0 |
| CE3 | 99.998 | 0 | 0 | 0.002 |
| CE4 | 99.995 | 0 | 0 | 0.005 |

The inventive and comparative examples were tested using 316L stainless steel coupons that were purchased from Alabama Specialty Products Inc, Munford, Alabama. The coupons had a flat rectangular shape (reference number: 316L SS UNS S31603), with a glass bead finish. Dimensions of the coupons were 2.54 centimeters (“cm”)×7.62 cm×1.58 millimeters and each had a weight of on average 22.96 g. One coupon was added to 100 g of each sample in the vial and then the mixtures were subjected to a temperature decrease from 80° C. to 60° C. by 5° C. increments every 24 hours, or a temperature decrease from 80° C. to 40° C. by 10° C. increments every 24 hours while in a furnace.

The total tocopherol content of the samples was measured before and after exposure to the decreasing temperature ranges. Tocopherol levels were measured using liquid chromatography with ultraviolet detection. The samples taken before and after the temperature exposure were heated in an oven at 75° C. for 1.5 to 2 hours until fully dissolved into a uniform liquid and lightly agitated. While still a uniform liquid, 0.5 g of sample was transferred to a glass vial and methanol was added up to a final weight of 5 g. A calibration curve was developed by preparing different standards. The standards were prepared by making a 1 wt % stock solution and doing a series of serial dilutions down to a concentration of 0.0005 wt % in methanol. AO1 and AO3 dissolved fairly easily at higher concentrations in the stock solution but AO2 did not and thus a 0.1 wt % concentration stock needed to be prepared for this antioxidant. Samples and standards were ran using the instrument conditions detailed in Table 2.

TABLE 2

| LC instrument: | Agilent 1260 Infinity Series Liquid Chromatograph | | |
|---|---|---|---|
| LC Column: | Zorbax Eclipse, 3.0 mm × 150 mm, 3.5 μm | | |
| Injection Volume: | 15 μL | | |
| Mobile Phase A: | Water | | |
| Mobile Phase B: | Methanol | | |
| Flow Rate: | 0.8 mL/min | | |
| | Time | | |
| Gradient: | (mins) | % A | % B |
| | 0 | 20 | 80 |
| | 2 | 0 | 0 |
| | 8 | 0 | 100 |

| | |
|---|---|
| Stop Time: | 8 mins |
| Post Time: | 5 mins |
| Column Temp: | 40° C. |
| DAD Wavelength: | 190-630 nm, extracted at 290 nm |

### Results

Tables 3 and 4 provides the results of the testing. The total tocopherol depletion represents the percent reduction of all tocopherols in the example after being subjected to the temperature change. In other words, the total tocopherol depletion is the difference between initial and final tocopherol contents divided by the initial tocopherol content and then multiplied by 100. The standard deviation (“Stdev”) was calculated using the replicate runs. Table 3 provides the percentage of total tocopherol depletion for a temperature decrease from 80° C. to 60° C. by 5° C. increments every 24 hours. Table 4 provides the percentage of total tocopherol depletion for a temperature decrease from 80° C. to 40° C. by 10° C. increments every 24 hours.

TABLE 3

| | Total | |
|---|---|---|
| | tocopherol | |
| Ex | depletion (%) | Stdev |
| IE1 | 37.59 | 17.26 |
| IE2 | 32.01 | 9.76 |
| CE1 | 66.60 | 2.39 |
| CE2 | 58.96 | 6.97 |
| CE3 | 86.22 | 0 |
| CE4 | 62.18 | 8.84 |

TABLE 4

| | Total | |
|---|---|---|
| | tocopherol | |
| Ex | depletion (%) | Stdev |
| IE1 | 36.38 | 6.44 |
| IE2 | 23.66 | 4.76 |
| CE1 | 68.20 | 8.17 |
| CE2 | 51.24 | 1.00 |
| CE3 | 63.48 | 7.13 |
| CE4 | 43.72 | 8.13 |

As is evident from Tables 3 and 4, IE1 and IE2 comprising a tocopherol blend where D-alpha tocopherol is less than 50 wt % of the combined D-alpha tocopherol, D-beta tocopherol, D-gamma tocopherol and D-delta tocopherol exhibit a much lower total tocopherol depletion as compared to CE1-CE4. Such a property is advantageous in that the reduced total tocopherol depletion means there is a greater amount of tocopherol remaining to prevent the formation of formaldehyde and other impurities. CE1 and CE2 demonstrate that glycol compositions comprising tocopherol blends where D-alpha tocopherol is greater than 50 wt % of the combined D-alpha tocopherol, D-beta tocopherol, D-gamma tocopherol and D-delta tocopherol exhibit nearly twice as much total tocopherol depletion which is expected to increase formaldehyde and impurity levels. Similarly to CE1 and CE2, CE3 and CE4 demonstrate that the glycol compositions comprising tocopherol blends where D-alpha tocopherol is the only tocopherol present exhibit nearly twice as much total tocopherol depletion which is expected to increase formaldehyde and impurity levels. A comparison of Table 3 and Table 4 illustrates that glycol compositions comprising a tocopherol blend where D-alpha tocopherol is less than 50 wt % of the combined D-alpha tocopherol, D-beta tocopherol, D-gamma tocopherol and D-delta tocopherol (e.g., IE1 and IE2) resist total tocopherol depletion across different temperature ranges and under different cooling rates.

1. A glycol composition, comprising:
a polyethylene glycol; and
a tocopherol blend comprising D-alpha tocopherol and one or more of D-beta tocopherol, D-gamma tocopherol and D-delta tocopherol, wherein the D-alpha tocopherol is less than 50 wt % of the combined D-alpha tocopherol, D-beta tocopherol, D-gamma tocopherol and D-delta tocopherol.

2. The glycol composition of claim 1, wherein the glycol is 80.000 wt % to 99.999 wt % of the total weight of the glycol composition.

3. The glycol composition of claim 1, wherein the glycol is a polyalkylene oxide having a weight average molecular weight of 400 g/mol to 10,000,000 g/mol as measured according to gel permeation chromatography.

4. The glycol composition of claim 3, wherein the glycol is a polyalkylene oxide having a weight average molecular weight of 2,000 g/mol to 5,000 g/mol as measured according to gel permeation chromatography.

5. (canceled)

6. The glycol composition of claim 1, wherein the glycol composition comprises 0.01 wt % or less of the tocopherol blend based on the total weight of the glycol composition.

7. The glycol composition of claim 6, wherein the glycol composition comprises 0.001 wt % or greater of the tocopherol blend based on the total weight of the glycol composition.

8. The glycol composition of claim 1, wherein the D-alpha tocopherol is 0 wt % to 40 wt % of the combined D-alpha tocopherol, D-beta tocopherol, D-gamma tocopherol and D-delta tocopherol.

9. The glycol composition of claim 8, wherein the D-alpha tocopherol is 10 wt % to 30 wt % of the combined D-alpha tocopherol, D-beta tocopherol, D-gamma tocopherol and D-delta tocopherol.

10. A formulation, comprising:
water; and
0.1 wt % to 99 wt % of the glycol composition of any one of claims 1-9 based on the total weight of the formulation.
